(19) United States
(12) Patent Application Publication
(10) Pub. No.: US 2010/0029788 A1
(43) Pub. Date: Feb. 4, 2010

(54) WET EDIBLE PEARLESCENT FILM COATINGS

(76) Inventor: John Pelesko, Roselle, NJ (US)

Correspondence Address: MICHAEL BEST & FRIEDRICH LLP, 100 E WISCONSIN AVENUE, Suite 3300, MILWAUKEE, WI, 53202, US

(21) Appl. No.: 12/443,028

(22) PCT Filed: Sep. 28, 2007
(86) PCT No.: PCT/US07/79967

§ 371 Date: Apr. 3, 2009

Related U.S. Application Data
(60) Provisional application No. 60/827,628, filed on Sep. 29, 2006.

(51) Int. Cl. A61K 47/34; A61K 47/30; A61K 47/26

(52) U.S. Cl. 514/772.7; 514/782; 514/781

(57) ABSTRACT

Provided herein are wet powder compositions which include a pearlescent pigment, a film-forming edible polymer and from about 1% to about 30% by weight water. The wet powder compositions may be used to make pearlescent film coating suspensions which include a pearlescent pigment, a film-forming polymer, a plasticizer and from about 70% to about 95% water. The pearlescent film coating suspensions may be used to coat edible products, such as pharmaceuticals, confectionary products and food products. Also disclosed are methods of making the wet powder compositions and the pearlescent film coating suspensions.

### CROSS-REFERENCE TO RELATED APPLICATIONS

This application claims priority to U.S. Provisional Application No. 60/827,628, filed Sep. 29, 2006, which is incorporated herein by reference in its entirety.

### FIELD OF INVENTION

This invention relates to edible film-forming or film coating compositions for use in the film coating of pharmaceuticals, confectionary and food products. The invention also relates to methods of making wet powder edible film coatings and methods of applying such coatings to products.

### BACKGROUND

Over the years, considerable effort has been expended to increase the visual appeal of tablets, capsules and other products. Many pharmaceutical manufacturers attempt to establish brand identity for their newly-approved products by altering the shapes, colors, etc. of the dosage forms. Consumers develop greater brand loyalty for distinctively appearing products as compared to those containing the same active ingredient in an unremarkable appearance, i.e. a white compressed tablet.

### SUMMARY

In one aspect, the invention may provide compositions comprising a wet mixture of film-forming polymers, pearlescent pigments and from about 1% to about 30% water. The compositions may also optionally include a plasticizer. The compositions are suitably edible and include non-toxic, edible, or food-grade polymers, pearlescent pigments and optionally plasticizers. The compositions may optionally contain edible pigments, dyes and natural colorants.

In another aspect, the invention may provide methods of making wet powder edible film coating compositions by mixing polymers, pearlescent pigments and optionally plasticizers and adding from about 1% to about 30% by weight water in atomized form such that the pigment-polymer blend is agglomerated and forms a film-forming composition.

In a further aspect, the invention may provide a pearlescent film-coating composition containing a pearlescent pigment, a film-forming polymer, a plasticizer and from about 70% to about 95% water by weight.

In yet another aspect, the invention may provide methods of coating products including pharmaceuticals, confectionary and food products by applying the pearlescent film-coating compositions to the products. The products may be coated in a single step.

In a still further aspect, the invention may provide edible products coated with the pearlescent film coatings and pharmaceutical, confectionary and food products comprising these edible food products.

### DETAILED DESCRIPTION

Before any embodiments of the invention are explained in detail, it is to be understood that the invention is not limited in its application to the details set forth in the following description. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

As used in this application, the singular forms “a,” “an,” and “the” include plural referents unless the content clearly dictates otherwise. Thus, for example, reference to a composition containing “a plasticizer” includes a mixture of two or more plasticizers. It should also be noted that the term “or” is generally employed in its sense including “and/or” unless the content clearly dictates otherwise.

All publications, patents and patent applications referenced in this specification are indicative of the level of ordinary skill in the art to which this invention pertains. All publications, patents and patent applications are herein expressly incorporated by reference to the same extent as if each individual publication or patent application was specifically and individually indicated by reference. In case of conflict between the present disclosure and the incorporated patents, publications and references, the present disclosure should control.

It also is specifically understood that any numerical value recited herein includes all values from the lower value to the upper value, i.e., all possible combinations of numerical values between the lowest value and the highest value enumerated are to be considered to be expressly stated in this application. For example, if a concentration range is stated as 1% to 50%, it is intended that values such as 2% to 40%, 10% to 30%, or 1% to 3%, etc., are expressly enumerated in this specification. If a concentration range is “at least 5%,” it is intended that all percentage values up to and including 100% are also expressly enumerated. These are only examples of what is specifically intended.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximation, the numerical values set forth in the specific examples are reported as precisely as possible. Any numerical value, however, inherently contain certain errors necessarily resulting from the standard deviation found in their respective testing measurements.

Provided herein are powder mixtures which are useful in preparing film coatings. The film coatings may have pearlescent qualities and are typically applied as suspensions to orally ingestible products such as pharmaceuticals (e.g., compressed tablets), confectionary products and other food products using pan coating or spraying techniques well-known to those of ordinary skill in the art. The wet powder pearlescent film-forming mixtures include a polymer, a pearlescent pigment and optionally a plasticizer. In one aspect, the invention may achieve a glossy, pearlescent coating in a single step without resorting to adding starches or other additives to the coating.

Powdered pearlescent pigment particles are first blended or mixed with a film-forming, water-soluble or water-dispersible, edible polymer to form a pearlescent pigment-polymer blend. Water is then applied onto the pearlescent pigment-polymer blend to produce a film-forming composition containing between approximately 1% and approximately 30% by weight of water. Suitably, the composition contains from about 2% to about 10% water by weight, more suitably from about 4% to about 9% water by weight. In one embodiment, the water may be applied in atomized form. Plasticizers may be added to the pearlescent pigment-polymer blend or can be added with or after the addition of water to form the film-forming compositions. The resulting compositions may be used to coat products such as pharmaceuticals, confectionary and food products with a pearlescent coating.

The film-forming, edible polymer component can be water-soluble or water-dispersible. Useful polymers of this type include, but are not limited to, methyl cellulose, hydroxypropyl methyl cellulose, hydroxypropyl cellulose, polyvinyl pyrrolidone, vinyl acetate/vinyl pyrrolidone copolymers (such as Plasdone S630® available from ISP Corp.), polyethylene glycol, polyvinyl alcohol/polyethylene glycol (such as Kollicoat IR® available from BASF Corp.) and natural gums such as gum tragacanth, gum acacia and xanthan gums. Mixtures or combinations of such polymers may also be used. The polymer used may have an average particle size from about 10 μm to about 200 μm, particularly from about 20 μm to about 180 μm, and more particularly from about 50 μm to about 150 μm.

The use of a pearlescent pigment confers the ability to impart improved pearlescence to the surface of edible articles. The pearlescent pigment should be capable of meeting all government approved requirements for human consumption if intended for use as a food coating. In one embodiment, the pearlescent pigment comprises a micaceous pearlescent pigment, such as those containing mica coated with titanium dioxide, iron oxide, and combinations thereof. Examples of pearlescent pigments include, but are not limited to, those available under the trade name Candurin® from Merck KGaA and those set forth in PCT publication No. WO 00/03609, the entire disclosure of which is incorporated herein by reference. A non-limiting list of suitable Candurin pearlescent pigment products include the following: silver fine, silver sheen, silver luster, sparkle silvers, gold shimmer, red shimmer, blue shimmer, green shimmer, gold sheen, light gold, gold luster, brown amber, orange amber, red amber, red luster, and red sparkle. Other edible pearlescent pigments known to those of skill in the art may also be used.

To prepare coating suspensions for use in coating products, the film-forming compositions are mixed with water and optionally a plasticizer. The coating suspensions/dispersions so prepared may be applied immediately to the products without a waiting period, such as overnight, as is required with certain prior art systems. Examples of plasticizers include, but are not limited to, at least one of acetylated monoglycerides (e.g., Myvacet®, produced by Kerry Bio-Sciences), polyethylene glycol 400, glycerin, propylene glycol, glycerine triacetate, triethyl citrate, triacetin, tributyl citrate, diethyl phthalate and combinations, and may be used in formulating coating suspensions from the wet film-forming compositions of the invention. The plasticizer may be incorporated into the wet film-forming compositions of the invention so that the resulting compositions may be simply added to water in order to form useful coating suspensions for coating products.

The compositions may optionally include additional powdered pigment components. Any FDA approved edible natural or synthetic colorant may be employed. Useful pigments include, but are not limited to, at least one of FD&C and D&C dyes, FD&C and D&C lakes, titanium dioxide, iron oxides, talc, alumina, silica, natural colorants and combinations thereof. The powdered pigment particles may be combinations of a lake, a dye, and titanium dioxide or iron oxide. The wet powder compositions may include from about 0.5% to about 20% pigment, particularly from about 0.5% to about 10%, and more particularly from about 1% to about 8%.

Useful wet powder, edible, pearlescent film-forming compositions for coating products including pharmaceutical tablets, capsules, confectionary products and food products may be formulated by mixing pearlescent pigment particles with a film-forming, water-soluble or water-dispersible, edible polymer and applying water onto the pearlescent pigment-polymer blend. In one embodiment, the water is applied in atomized form. When formulated in this manner, the resulting film-forming compositions have been found capable of tolerating up to approximately 30% by weight water while remaining capable of forming a stable suspension upon dilution with additional water. Also, the film-forming compositions may not have a tendency to clump or contain fish eyes and, when combined with additional water and a plasticizer, provide a smooth, uniform coating for pharmaceutical tablets, capsules and the like. The method does not require addition of a starch to form film coating dispersions/suspensions. In addition, this method does not require heating of the mixture and the time required from initial mixing to coating products with the film-forming dispersions/suspensions is short (e.g., 30 minutes to 1 hour) in comparison to other methods.

In one embodiment, after the pearlescent pigment and polymer have been blended until uniform, the desired amount of water is added with intense mixing so that the water is applied to the pigment-polymer blend in atomized form. This can be accomplished, for example, by blending the powdered pigment and polymer in a high speed, high shear powder mixing device, such as a PK Blender or a Littleford FM 130 Blender, and adding the water with intense mixing by fine atomization into the blender chamber where the powdered pigment and polymer are mixed. The water may be added slowly and the resulting water-containing blend or composition is mixed intensely after addition of the water.

It is believed that the addition or application of water onto the pearlescent pigment-polymer blend in atomized form preconditions the polymer and avoids clumping or the formation of fish eyes when the compositions are added to water to form coating suspensions. Thus, by applying the water in atomized form, the water becomes uniformly distributed over all polymer particles, the pearlescent pigment particles adhere to the polymer particles and the polymer is preconditioned so that hydration of the polymer is expedited when the pearlescent pigment-polymer blend is dispersed in water to form a coating suspension. Accordingly, preconditioning of the blend of pearlescent pigment and polymer causes the blend to readily disperse and dissolve in water to form the desired coating suspension without deleterious lumping or agglomeration. Moreover, smooth coating suspensions can be prepared and used immediately or within 30 minutes for coating products.

The amount of polymer included in the powder mixtures may be from about 10% to about 85% by weight. The polymer ranges may also be from about 25% to about 85%, from about 35% to about 80%, or from about 50% to about 75%.

The powder mixtures include (by weight) from about 0.5% to about 20% pearlescent pigment, particularly from about 0.5% to about 10%, and more particularly from about 0.5% to about 8%. It will be understood, however, that the amount of pearlescent pigment employed in the powder mixtures of the invention is dependent on the opacity of the specific pigment being utilized and whether other pigments are being added to the mixture. The amount is based on what is sufficient or effective to impart an improved pearlescent outer coating to the surface of the product to be coated.

The powder mixtures may also include a plasticizer which may range from about 0.75% to about 20%, particularly from about 0.75% to about 17%, particularly from about 0.9% to about 16%, and more particularly from about 1% to about 15%. The amount of plasticizer will vary depending on which plasticizer is utilized. Alternatively, the plasticizer may be added after the atomized water in an equivalent ratio based on the weight of the polymer mixture.

In one embodiment, the wet powder mixtures may comprise about 40% to about 80% by weight polymer, about 5% to about 20% by weight plasticizer, about 0% to about 10% by weight dye or pigment, about 0.5% to about 8% by weight pearlescent pigment and about 2% to about 10% by weight water.

It has been found that film-forming compositions prepared in this manner may contain up to approximately 30% by weight of water and yet remain capable of forming a stable suspension useful for coating tablets and the like upon dilution with additional water. It should be noted that where FD&C lakes are employed as pigments, they may contain up to about 15% by weight of water and that the polymer component may likewise contain up to about 4% to about 6% by weight of water. The water added through the practice of this invention is additional water over and above that contained in the pigment and polymer components and, as stated, may range from about 1% to about 30% by weight, about 2% to 10% by weight and about 4% to about 9% by weight. The wet powder compositions may then be mixed with water to form film coating suspensions/dispersions that are useful to coat products. The amount of water added to the wet powdered compositions to form the dispersions may be up to about 95% by weight. Suitably, the dispersions are made by adding from about 70% to about 95% by weight water, suitably about 75% to about 95% water, more suitably from about 80% to about 90% water. This includes 80-95% water dispersions.

The film-forming compositions of the invention are shelf-stable for extended periods of time without the use of preservatives and are not prone to settling or other breakdowns. Further, it is believed that such compositions remain free from bacteria formation such as may be normally caused by the solvent in liquid pigment dispersions.

In still further embodiments of the invention, there are provided orally-ingestible substrates having a pearlescent film coating as well as methods of coating ingestible substrates using the compositions described herein. As will be described in the Examples below, the methods include applying the pearlescent film-forming suspensions described herein to a surface of an orally ingestible substrate or product. The pearlescent film-forming suspensions can be applied by any method known to those of skill in the art including, but not limited to, pan coating, spray coating, and use of a Wurster column. The amount of pearlescent film-forming suspension applied will depend upon several factors, including the product to be coated, the amount and color of the pearlescent pigment included in the suspensions, the apparatus employed to apply the coating, and other factors known to those of skill in the art. In some embodiments of the invention, the products will be coated to a theoretical weight gain of from about 0.25% to about 20% by weight and particularly from about 0.25% to about 5.0%. Suitably, the theoretical weight gain is from about 0.5% to about 4.0% and more suitably, the theoretical weight gain is from about 1.0% to about 3.0% or from about 1.0% to about 4.0% by weight of said product.

After the coating is applied to a product, the gloss, smoothness, and slip may be measured and compared using methods known to those of skill in the art. For example, gloss and smoothness testing may be performed using a Surface Analyzer available from Tricor Systems, Model 806 or Glossmeters available from Paul N. Gardner Equipment Co. In addition, slip may be measured using Texture Technology Equipment Model TA.XT2i.

The following examples are provided to assist in a further understanding of the invention. The particular materials and conditions employed are intended to be further illustrative of the invention and are not limiting upon the reasonable scope of the invention.

### EXAMPLES

### Example 1

### Blue Pearlescent Film Coating Formulation

The formulation was made using the following constituents:

| | |
|---|---|
| Spectracel 5 FG (HPMC) | 76.6 parts by weight |
| Candurin Silver Lustre | 6.0 parts by weight |
| FD&C Blue No. 1 dye | 0.04 parts by weight |
| Myvacet | 13.0 parts by weight |
| Water | 4.0 parts by weight |

The formulations were prepared by adding all the solid, powder components to a high speed, high agitation blender and blending until uniform, approximately 1-10 minutes. Water (4 grams, in the above formulation) was atomized and sprayed onto the agitated powder bed and finally the plasticizer (13 grams in the above formulation) was atomized and sprayed onto the agitated powder bed. The water was added over a period of approximately 1-5 minutes as was the plasticizer. The resultant formulation was used to spray-coat placebo tablets after preparing a 13% dispersion of the formulation by adding 87% by weight additional water to the formulation with agitation.

### Example 2

### Formulations for Pearlescent Coated Tablets in a Variety of Colors

The formulations shown in Table 1 (units of measurement are grams) were made by adding the individual ingredients to the water and mixing as described in Example 1 to form a pigment-polymer-plasticizer blend. The resultant dispersions were used to coat tablets by spray coating. To test the adequacy of coverage, several dark cores were included. Each of the formulations produced a film coating on the tablets with good coverage and a pearlescent appearance. Good coverage is defined as one pleasing to the eye, consisting of a uniform, unbroken film, uniform in shade and color with no evidence of the original tablet surface showing. In addition, tablets may be tested for gloss, smoothness and slip. The added dye or lake color was evenly distributed on the tablet with no fish eyes or clumping of the film-coating.

TABLE 1
Formulations containing various dyes, a lake & titanium dioxide in combination with a
pearlescent pigment.

| | Experiment No. | | | | | | | | |
|---|---|---|---|---|---|---|---|---|---|
| | 033- | 033- | 033- | 033- | 033- | 033- | 033- | 033- | 033- |
| | 115-1 | 115-2 | 115-3 | 115-4 | 115-5 | 115-6 | 115-7 | 115-8 | 115-9 |
| 10% HPMC | 45 | 45 | 45 | 45 | 45 | 45 | 45 | 45 | 45 |
| PEG 400 | 0.9 | 0.9 | 0.9 | 0.9 | 0.9 | 0.9 | 0.9 | 0.9 | 0.9 |
| Candurin Silver | 0.5 | 0.5 | 0.5 | 0.5 | 0.5 | 0.5 | 0.5 | 0.5 | 0.1 |
| Lustre | | | | | | | | | |
| FDC Blue 1 Dye | 0.02 | — | — | — | — | — | — | — | — |
| FDC Blue 2 Dye | — | 0.02 | — | — | — | — | 0.2 | 0.005 | 0.02 |
| FDC Yellow 5 Dye | — | — | 0.02 | — | — | — | — | 0.015 | — |
| FDC Yellow 6 Dye | — | — | — | 0.02 | — | — | — | — | — |
| FDC Red 40 Dye | — | — | — | — | 0.02 | — | — | — | — |
| FDC Blue 1 Lake | — | — | — | — | — | 0.05 | — | — | — |
| Titanium dioxide | — | — | — | — | — | — | 0.5 | — | — |

### Example 3

### Formulations Containing Different Pearlescent Pigments

The formulations shown in Table 2 (units of measurement are grams) were made by blending the indicated amounts of each constituent as described in Example 2 above. The resultant dispersions were used to coat tablets by spray coating. The silver and gold luster pearlescent pigments gave the tablet the indicated pearlescent color and provided full coverage of the tablet color. The gold shimmer provided a lighter pearlescent effect and did not provide as much coverage of the tablet color as compared to the luster pigments. The red amber gave a red pearlescent color to the tablet and provided similar coverage to the silver and gold luster pigments. Spectracel 5 FG HPMC is available from Sensient Colors, Inc. (St. Louis, Mo.).

TABLE 2
Formulations containing various pearlescent pigments.

| | Experiments No. | | | |
|---|---|---|---|---|
| | 033-172-1 | 033-172-2 | 033-172-3 | 033-172-4 |
| Spectracel 5 FG HPMC | 10 | 10 | 10 | 10 |
| Candurin Silver Lustre | 1 | — | — | — |
| Candurin Gold Lustre | — | 1 | — | — |
| Candurin Gold Shimmer | — | — | 1 | — |
| Candurin Red Amber | — | — | — | 1 |
| PEG 400 | 2 | 2 | 2 | 2 |
| water | 87 | 87 | 87 | 87 |

### Example 4

### Comparison of Addition of Dyes and Lakes to Supplement Pearlescence

The formulations shown in Table 3 (units of measurement are grams) were made by blending the indicated amounts of each constituent as described in Example 2 above. The resultant dispersions were used to coat tablets by spray coating. Both dyes and lakes may be added to the pearlescent film-forming compositions to add color distinctiveness to the resulting tablets. The dye and lake provided similar color value or color strength.

TABLE 3
Formulations using dyes or lakes.

| | Experiments No. | | |
|---|---|---|---|
| | 033-173-1 | 033-173-2 | 033-173-3 |
| Spectracel 5 FG HPMC | 10 | 10 | 10 |
| PEG 400 | 2 | 2 | 2 |
| Water | 86.98 | 86.98 | 86.98 |
| FDC Red No. 3 Dye | 0.02 | — | — |
| FDC Yellow No. 5 Dye | — | 0.02 | — |
| FDC Yellow No. 5 lake | — | — | 0.06 |
| Candurin Gold Lustre | 1 | 1 | 1 |

### Example 5

### Comparison of the Effects of Various Plasticizers on Pearlescence

The formulations shown in Table 4 (units of measurement are grams) were made by blending the indicated amounts of each constituent as described in Example 2 above. The resultant dispersions were used to coat tablets by spray coating. The formulations contain different plasticizers, namely acetylated monoglycerides (e.g., Myvacet®), polyethylene glycol 400, triethyl citrate (TEC), and triacetin. Tablets coated with the dispersions containing different plasticizers all had similar color and pearlescence. Myvacet® provided a more vibrant color than the others and the coated tablets were slippery.

TABLE 4
Formulations containing various plasticizers.

| | Experiments No. | | | | |
|---|---|---|---|---|---|
| | 033-174-1 | 033-174-2 | 033-174-3 | 033-174-4 | 033-174-5 |
| Spectracel 5 | 10 | 10 | 10 | 10 | 10 |
| FG HPMC | | | | | |
| Candurin | 1 | 1 | 1 | 1 | 0.05 |
| Silver Lustre | | | | | |
| FDC Blue | 0.02 | 0.02 | 0.02 | 0.02 | 0.02 |
| No. 1 dye | | | | | |
| Water | 86.98 | 86.98 | 86.98 | 86.98 | 86.98 |
| PEG 400 | 2 | — | — | — | — |
| Myvacet | — | 2 | — | — | 2 |
| Triethyl | — | — | 2 | — | — |
| citrate | | | | | |
| Triacetin | — | — | — | 2 | — |

### Example 6

### Use of Pearlescent Film Coatings as a Top Coat Over Black

The formulations shown in Table 5 (units of measurement are grams) were made by blending the indicated amounts of each constituent as described in Example 2 above. The resultant dispersions were used to coat tablets by spray coating. The formulations listed as 1-2 and 7-9 in Table 5 were used to coat tablets pre-coated with Spectrablend Black 88906N020 available from Sensient Colors, Inc. (St. Louis, Mo.) and formulations 3-6 in Table 5 were pre-coated with the indicated amount of black iron oxide. Formulations 1 and 7 did not coat evenly at a 2.5-3% weight gain, but improved and provided a gold pearlescence effect when coated at 5% weight gain. Formulations 2 and 8 provided a uniform coating and a copper shine effect. Formulation 9 provided an even coating with a silver pearl effect. Formulations 3 and 4 provided an even, uniform, and consistent coating on both tablets and capsules. These formulations created a gold pearl effect at 2.5-3% weight gain. Formulations 5 and 6 provided a consistent and uniform coating on tablets. These formulations created a silver effect at 2.5-3% weight gain.

TABLE 5
Formulations for provision of a pearlescent pigment top-coating over
black pre-coated tablets.

| | Experiment No. | | | | | | | | |
|---|---|---|---|---|---|---|---|---|---|
| | | 033- | 033- | 033- | 033- | 033- | 033- | 033- | 033- |
| | 033-175-1 | 175-2 | 175-3 | 175-4 | 175-5 | 175-6 | 175-7 | 175-8 | 175-9 |
| Spectracel 5 FG | 10 | 10 | 10 | 10 | 10 | 10 | 8 | 8 | 8 |
| HPMC | | | | | | | | | |
| Hydroxypropyl- | — | — | — | — | — | — | 2 | 2 | 2 |
| cellulose | | | | | | | | | |
| Candurin Silver | — | — | — | — | 1 | 1 | — | — | 1 |
| Lustre | | | | | | | | | |
| Candurin Gold | 1 | — | 1 | 1 | — | — | 1 | — | — |
| Shimmer | | | | | | | | | |
| Candurin Red | — | 1 | — | — | — | — | — | 1 | — |
| Amber | | | | | | | | | |
| PEG 400 | 2 | 2 | 2 | 2 | 2 | 2 | 2 | 2 | 2 |
| Water | 87 | 87 | 86.9 | 86.7 | 86.9 | 86.7 | 87 | 87 | 87 |
| Black Iron Oxide | — | — | 0.1 | 0.3 | 0.1 | 0.3 | — | — | — |

### Example 7

### Formulation of an Orange Pearlescent Film-Forming Composition

The formulations shown in Table 6 (units of measurement are grams) were made by blending the indicated amounts of each constituent as described in Example 1 above. The specific color desired for a use can be made by mixing various dyes. Table 6 provides two formulations for an orange color pearlescent film-forming composition. Tablets were coated at 2.5% weight gain and produced a distinctive colored tablet with a slippery, glossy finish. The formulation made using Myvacet® was considered superior because of increased smoothness of the finish.

TABLE 6
Orange pearlescent film-forming compositions.

Experiments No.

| | 033-176 | 033-183 |
|---|---|---|
| Spectracel 5 FG HPMC | 74.9 | 74.9 |
| Candurin Silver Lustre | 8 | 8 |
| FDC Yellow No. 6 dye | 0.7 | 0.7 |
| FDC Red No. 40 dye | 0.4 | 0.4 |
| PEG 400 | 12 | — |
| Myvacet | — | 12 |
| water | 4 | 4 |

### Example 8

### Formulation of a Pearlescent Film-Forming Composition with an Alternative Polymer

The formulations shown in Tables 7 and 8 (units of measurement are grams) were made by blending the indicated amounts of each constituent as described in Example 2 above. The resultant dispersions were used to coat tablets by spray coating. The formulations were made to analyze the effect of using different polymers on the final coating. Table 7 made use of Plasdone S630 VP/VA® available from ISP Corp. and produces a product with improved surface appearance. Table 8 shows the formulation made using Kollicoat IR® from BASF Corp. Use of this polymer produced a coating with poor gloss and poor surface appearance. Tablets were coated at 2.0-2.5% weight gain.

TABLE 7
Use of an alternative polymer, Plasdone S630 VP/VA (ISP Corp.)

| | Experiment No. |
|---|---|
| | 033-185 |
| Shade | white |
| Spectracel 5 FG | 18.9 |
| Maltodextrin M040 | 5.6 |
| Candurin Silver Lustre | 3 |
| FDC Blue No. 1 Dye | 0.1 |
| water | 365 |
| Myvacet | 5 |
| Plasdone S-630 | 2.4 |

TABLE 8
Use of another alternative polymer,
Kollicoat IR (BASF Corp.)

| | Experiment No. |
|---|---|
| | 038-21 |
| Shade | blue |
| Kollicoat IR | 24 |
| Candurin Silver Lustre | 1 |
| FDC Blue No. 1 Dye | 0.02 |
| water | 74.98 |

### Example 9

### Alternative Formulations of Pearlescent Film-Forming Compositions

The formulations shown in Table 9 (units of measurement are grams) were made by blending the indicated amounts of each constituent as described in Example 1 (except 038-57, 038-61-1, 038-62-2 which were made as described in Example 2) above. The formulations shown in Table 10 were made as described in Example 2. Tablets were coated at 2.0-2.5% weight gain with a 13% dispersion of the formulations. The formulations demonstrated a wide variety of distinct colors and gloss levels achieved by altering the formulations.

TABLE 9
The following are various formulations

| | Experiment No. | | | | | | |
|---|---|---|---|---|---|---|---|
| | 038-55 | 038-56 | 038-57 | 038-61-1 | 038-61-2 | 038-62-1 | 038-62-2 |
| Shade | Red | Dark Green | Beige | Beige | Beige | white | white |
| Spectracel 5 FG | 69.38 | 71.7 | 37.25 | — | — | — | — |
| Spectracel 6 FG | — | — | — | 47.4 | 47.4 | 68.385 | 68.5 |
| Candurin Silver Lustre | 8 | 8 | 4 | 4.8 | 4.8 | 8 | 8 |
| Myvacet | 12 | 12 | 6 | 7.2 | 7.2 | 9 | 11.76 |
| FDC Red 40 Dye | 2.65 | — | — | — | — | — | — |
| FDC Red 3 Dye | 3.97 | — | — | — | — | — | — |
| FDC Blue 1 Dye | — | 0.7 | — | — | — | — | — |
| FDC Yellow 5 Dye | — | 3.6 | — | — | — | — | — |
| caramel | — | — | 0.5 | 0.6 | 0.3 | — | — |
| titanium dioxide | — | — | — | — | — | 7.82 | 7.82 |
| PEG 400 | — | — | — | — | — | 2.76 | — |
| Polysorbate 80 | — | — | — | — | — | 0.115 | — |
| water | 4 | 4 | 452 | 440 | 440 | 3.92 | 3.92 |

TABLE 10
The following are various formulations

| | Experiment No. | | | | | | |
|---|---|---|---|---|---|---|---|
| | 040-07-1 | 040-07-2 | 040-07-3 | 040-07-4 | 040-15-1 | 040-15-2 | 040-15-3 |
| Shade | Red | Red | Red | Red | Silver | Silver | Silver |
| 10% Spectracel 6 FG | 94 | 95 | 95.55 | 95.7 | — | — | — |
| Spectracel 6 FG | — | — | — | — | 75.836 | 75.925 | 75.25 |
| Spectracel 5 FG | — | — | — | — | — | — | — |
| PEG 400 | 2 | — | — | — | — | — | — |
| Myvacet | — | 2 | 2 | 2 | 12 | 12 | 12 |
| Candurin Pearl Lustre | 2 | 2 | 2 | 2 | 8 | 8 | 8 |
| FDC Red 40 Dye | 0.8 | 0.4 | 0.4 | 0.3 | — | — | — |
| FDC Red 3 Dye | 1.2 | 0.6 | 0.05 | — | — | — | — |
| FDC Blue 2 Lake | — | — | — | — | 0.1 | 0.4 | 0.4 |
| 09918 | | | | | | | |
| FDC Yellow 6 Lake | — | — | — | — | 0.032 | 0.015 | 0.15 |
| 09610 | | | | | | | |
| FDC Red 40 Lake | — | — | — | — | 0.032 | 0.02 | 0.2 |
| 09313 | | | | | | | |
| water | — | — | — | — | 4 | 4 | 4 |

Note
Experiments 040-15-1, 040-15-2 & 040-15-3 are coated from a 13% dispersion.

### Example 10

### Formulations Comprising Titanium Dioxide

The formulations shown in Table 11 (units of measurement are grams) were made by blending the indicated amounts of each constituent as described in Example 1 above. Tablets were coated at 2.0-2.5% weight gain. With increasing titanium dioxide opacity increases but the pearlescent effect is diminished. All tablets were coated with 13% dispersions of the formulations shown.

TABLE 11
The effect of increased additions of titanium dioxide

| | Experiment No. | | | | | | |
|---|---|---|---|---|---|---|---|
| | 040-16-1 | 040-16-2 | 040-16-3 | 040-16-4 | 040-16-5 | 040-16-6 | 040-16-7 |
| Shade | white | white | white | white | white | white | white |
| Spectracel 5 FG | 76 | 75 | 72 | 68 | 80 | 79 | 76 |
| Candurin Silver | 8 | 8 | 8 | 8 | 4 | 4 | 4 |
| Lustre | | | | | | | |
| PEG 400 | 12 | 12 | 12 | 12 | 12 | 12 | 12 |
| water | 4 | 4 | 4 | 4 | 4 | 4 | 4 |
| titanium dioxide | — | 1 | 4 | 8 | — | 1 | 4 |

### Example 11

### Additional Formulations

The formulations shown in Table 12 (units of measurement are grams) were made by blending the indicated amounts of each constituent as described in Example 1 above. Tablets were coated at 2.0-2.5% weight gain. All tablets were coated with 13% dispersions of the formulations shown.

TABLE 12
Additional formulations.

| | Experiment No. | | | | |
|---|---|---|---|---|---|
| | 040-17-1 | 040-17-2 | 040-18-1 | 040-18-2 | 040-18-3 |
| Shade | pink | pink | peach | peach | peach |
| Spectracel | 75.79 | 74.79 | 75.95 | 75.945 | 75.966 |
| 5 FG | | | | | |
| Candurin | 8 | 8 | 8 | 8 | 8 |
| Silver Lustre | | | | | |
| PEG 400 | 12 | 12 | 12 | 12 | 12 |
| FDC Red 40 | 0.1 | 0.1 | — | — | — |
| Dye | | | | | |
| FDC Red 40 | 0.11 | 0.11 | 0.024 | 0.03 | 0.014 |
| Lake 09310 | | | | | |
| FDC Yellow | — | — | 0.026 | 0.025 | 0.02 |
| 6 Dye | | | | | |
| Water | 4 | 4 | 4 | 4 | 4 |
| Titanium | — | 0.1 | — | — | — |
| dioxide | | | | | |

### Example 12

### Formulations Comprising Natural Colorants

The formulations shown in Table 13 (units of measurement are grams) were made by blending the indicated amounts of each constituent as described in Example 1 above. Tablets were coated at 2.0-2.5% weight gain. While shade increases in intensity with additional colorant, the pearlescent effect is lessened in each case. All tablets were coated with 13% dispersions of the formulations shown.

TABLE 13
The following experiments were run to show the effect of natural colorants with
pearlescent pigment

| | Experiment No. | | | | | | | | | | |
|---|---|---|---|---|---|---|---|---|---|---|---|
| | 040- | 040- | 040- | 040- | 040- | 040- | 040- | 040- | 040- | 040- | 040- |
| | 20 | 21-1 | 21-2 | 21-3 | 21-4 | 21-5 | 21-6 | 21-7 | 21-8 | 19-1 | 19-2 |
| Shade | pink | yellow | yellow | yellow | yellow | red | red | green | green | green | green |
| Spectracel 5 | 75.76 | 75 | 71 | 75 | 71 | 75 | 71 | 75 | 71 | 75.7 | 74.9 |
| FG | | | | | | | | | | | |
| PEG 400 | 12 | 12 | 12 | 12 | 12 | 12 | 12 | 12 | 12 | 12 | 12 |
| Candurin | 8 | 8 | 8 | 8 | 8 | 8 | 8 | 8 | 8 | 8 | 8 |
| Silver Lustre | | | | | | | | | | | |
| water | 4 | 4 | 4 | 4 | 4 | 4 | 4 | 4 | 4 | 4 | 4 |
| carmine | 0.24 | — | — | — | — | — | — | — | — | — | — |
| annatto | — | 1 | 5 | — | — | 1 | 5 | — | — | — | — |
| riboflavin | — | — | — | 1 | 5 | — | — | — | — | — | — |
| chlorophyllin | — | — | — | — | — | — | — | 1 | 5 | 0.3 | 0.6 |
| titanium | — | — | — | — | — | — | — | — | — | — | 0.5 |
| dioxide | | | | | | | | | | | |

### Example 13

### Formulations Comprising Triacetin as the Plasticizer

The formulations shown in Table 14 (units of measurement are grams) were made by blending the indicated amounts of each constituent as described in Example 1 above. Tablets were coated at 2.0-2.5% weight gain. Triacetin formulations were less pearlescent than similar formulations made with Myvacet as the plasticizer. Furthermore, the use of the lake form of the equivalent dyes resulted in less glossy coatings than when the dye form of the colorant was used. All tablets were coated with 13% dispersions of the formulations shown.

TABLE 14
The following formulations contain triacetin as the plasticizer

| | Experiment No. | | | | |
|---|---|---|---|---|---|
| | 040-22-1 | 040-22-2 | 040-22-3 | 040-22-4 | 040-22-5 |
| Shade | light green | light green | light green | light green | light green |
| Spectracel 5 FG | 75.85 | 75.6 | 75.54 | 75.49 | 75.45 |
| Candurin Silver Lustre | 8 | 8 | 8 | 8 | 8 |
| water | 4 | 4 | 4 | 4 | 4 |
| FDC Blue 1 Lake 09903 | 0.05 | 0.2 | — | — | — |
| FDC Yellow 5 Lake 09609 | 0.1 | 0.2 | 0.2 | 0.2 | 0.2 |
| FDC Blue 2 Lake 09918 | — | — | 0.16 | 0.21 | 0.25 |
| FDC Yellow 6 Lake 09606 | — | — | 0.1 | 0.1 | 0.1 |
| triacetin | 12 | 12 | 12 | 12 | 12 |

### Example 14

### Formulations Comparing Various Plasticizers

The formulations shown in Table 15 (units of measurement are grams) were made by blending the indicated amounts of each constituent as described in Example 1 above. Tablets were coated at 2.0-2.5% weight gain. Formulations made with PEG400 as the plasticizer were less glossy and less slippery than when Myvacet was used as the plasticizer. All tablets were coated with 13% dispersions of the formulations shown.

TABLE 15
Formulations comparing PEG400 and Myvacet.

| | Experiment No. | | | | | | | |
|---|---|---|---|---|---|---|---|---|
| | 040-29 | 040-31 | 040-32 | 040-51 | 040-77-1 | 040-77-2 | 040-78 | 040-96 |
| Shade | peach | gold | silver | Dark | blue | blue | gray | gold |
| | | | | Green | | | | |
| Spectracel 5 | 75.966 | 74.532 | 76.287 | 71.7 | 75.5 | 75.5 | 75.965 | 76.287 |
| Fg | | | | | | | | |
| PEG 400 | 12 | 12 | 12 | — | — | — | — | — |
| Candurin | 8 | 7.5 | 7.5 | 8 | 8 | 8 | 7 | 7.5 |
| Silver Lustre | | | | | | | | |
| water | 4 | 4 | 4 | 4 | 4 | 4 | 4 | 4 |
| FDC Red 40 | 0.014 | — | — | — | — | — | — | — |
| Lake 09310 | | | | | | | | |
| FDC Yellow 5 | 0.02 | 1.885 | 0.19 | 3.6 | — | — | — | 0.19 |
| Dye | | | | | | | | |
| FDC Yellow 6 | — | 0.83 | 0.023 | — | — | — | — | 0.023 |
| Lake 09606 | | | | | | | | |
| Myvacet | — | — | — | 12 | 12 | 12 | 12 | 12 |
| FDC Blue 1 | — | — | — | 0.7 | — | — | — | — |
| Dye | | | | | | | | |
| FDC Blue 1 | — | — | — | — | 0.5 | 0.25 | 0.125 | — |
| Lake 09903 | | | | | | | | |
| FDC Blue 2 | — | — | — | — | — | 0.25 | — | — |
| Lake 09918 | | | | | | | | |
| FDC Yellow 5 | — | — | — | — | — | — | 0.58 | — |
| Lake 09733 | | | | | | | | |
| FDC Red 40 | — | — | — | — | — | — | 0.33 | — |
| Lake 09313 | | | | | | | | |

### Example 15

### Coating Confectionary or Food Products with Pearlescent Film Coating Suspensions

Formulations such as those described in Examples 1-14 may also be used to coat other products such as confectionary and food products. The confectionary or food products may be candy, more specifically, hard/shelled candy, such as chocolate or fruit-flavored candy. The confectionary products will be spray-coated with the pearlescent film coating suspensions made from the formulations as described above.

We claim:

1. A wet powder composition comprising a pearlescent pigment, a film-forming edible polymer and from about 1% to about 30% by weight water.

2. The composition of claim 1, wherein the polymer comprises at least one of methyl cellulose, hydroxypropyl methyl cellulose, hydroxypropyl cellulose, polyvinyl pyrrolidone, vinyl acetate/vinyl pyrrolidone copolymers, natural gums, gum tragacanth, gum acacia, xanthan gums and combinations thereof.

3. The composition of claim 1, wherein the polymer comprises hydroxypropyl methyl cellulose.

4. The composition of claim 1, comprising from about 10% to about 85% by weight polymer.

5. The composition of claim 1, wherein the polymer has an average particle size of from about 10 μm to about 190 μm.

6. The composition of claim 1, wherein the pearlescent pigment is edible.

7. The composition of claim 1, comprising from about 0.5% to about 10% pearlescent pigment.

8. The composition of claim 1, further comprising a powdered pigment particle.

9. The composition of claim 8, wherein the powdered pigment particle comprises at least one of FD&C dyes, D&C dyes, FD&C lakes, D&C lakes, titanium dioxide, iron oxides, talc, alumina, silica, natural colorants and combinations thereof.

10. The composition of claim 1, further comprising a plasticizer.

11. The composition of claim 10, comprising from about 0.75% to about 20% plasticizer.

12. The composition of claim 10, wherein the plasticizer comprises at least one of an acetylated monoglyceride, polyethylene glycol, glycerin, propylene glycol, glycerine triacetate, triethyl citrate, triacetin, tributyl citrate, diethyl phthalate and combinations thereof.

13. The composition of claim 10, wherein the plasticizer comprises polyethylene glycol 400.

14. The composition of claim 10, wherein the plasticizer comprises an acetylated monoglyceride.

15. The composition of claim 1, comprising from about 40% to about 80% by weight polymer, from about 5% to about 20% by weight plasticizer, from about 0.5% to about 8% by weight pearlescent pigment and from about 2% to about 20% water by weight.

16. A method of making a pearlescent film coating composition comprising:
a) mixing a pearlescent pigment and a film-forming edible polymer to form a pigment-polymer blend;
b) adding from about 1% to about 30% by weight water in atomized form to the pigment-polymer blend to form a film-forming composition.

17. The method of claim 16, wherein the polymer is hydroxypropyl methyl cellulose.

18. The method of claim 16, wherein (a) further comprises mixing a powdered pigment particle.

19. The method of claim 16, wherein (a) further comprises mixing a plasticizer.

20. The method of claim 16, further comprising
c) adding a liquid plasticizer in atomized form to the film-forming composition.

21. The method of claim 16, wherein the composition is capable of forming a suspension upon dilution with up to about 95% by weight water.

22. A pearlescent film coating suspension comprising a pearlescent pigment, a film-forming polymer, a plasticizer and from about 70% to about 95% water.

23. An edible product coated with the composition of claim 22.

24. A pharmaceutical, a confectionary or a food product comprising the edible product of claim 23.

25. The product of claim 23, wherein the composition of claim 22 comprises from about 0.25% to about 5.0% by weight of the product.

26. A method of coating an edible product comprising applying the composition of claim 22 to the surface of the edible product.
